(19) United States
(12) Patent Application Publication
(10) Pub. No.: US 2013/0142746 A1
(43) Pub. Date: Jun. 6, 2013

(54) PRESSURIZED DELIVERY OF HAIR STYLING GELS

(71) Applicants: Robert Demson, Cave Creek, AZ (US); Jennifer Harris, Phoenix, AZ (US)

(72) Inventors: Robert Demson, Cave Creek, AZ (US); Jennifer Harris, Phoenix, AZ (US)

(73) Assignee: The Dial Corporation, Scottsdale, AZ (US)

(21) Appl. No.: 13/308,655

(22) Filed: Dec. 1, 2011

(51) Int. Cl. A61K 8/00

(52) U.S. Cl. 424/70.1

(57) ABSTRACT

An aerated hair styling gel delivery system comprises a hair styling gel with entrained air stably packaged in and delivered from a bag-on-valve dispensing package. The hair gel remains lighter in density than hair styling gels packaged in conventional plastic cosmetic tubes or tubs.

### FIELD OF THE INVENTION

The present invention generally relates to pressurized packaging and aerosolized dispensing of gels, and more particularly relates to hair styling gels dispensed in the form of aerated gels from bag-on-valve packaging.

### BACKGROUND OF THE INVENTION

Hair styling compositions may be provided in a variety of physical forms, such as liquid, gel, mousse, or pomade, depending on the composition, styling/setting performance, consumer acceptance, branding, and marketing, amongst other variables. Particularly popular hair styling products include the hair spiking gels such as “Göt2b Glued” from Schwarzkopf & Henkel. Hair styling gels are customarily packaged in cosmetic tubes, plastic bottles with a dispensing orifice and hinged-lid closure, or in screw-cap jars. Hair styling gels are not readily found in any type of aerosolized packaging anywhere in the personal care market.

Many examples of hair styling gel compositions exist in the prior art. Some of the more interesting and relevant examples are found in U.S. Pat. Nos. 6,132,704 (Bhatt et al.); 5,985,294 (Peffly); 5,853,706 and 5,843,415 (Klar); 5,833,968 (Keil et al.); 5,098,699 (Hayama et al.); and 5,032,391 (Helioff et al.). Also of interest, and most particularly relevant as hair styling gel examples, are the compositions disclosed in U.S. Patent Application Publications 2009/0123405 (Sun et al.) and 2008/0102051 (Huynh et al.).

Some personal care gels, however, are routinely packaged in an aerosolized package. Post-foaming gels are well known and consist mostly of personal cleansing soap compositions and shaving gels. Post-foaming gels require the entrainment of a volatile gas into the composition prior to packaging in the aerosol can. After the gel is dispensed from the can, the volatile gasses liberate, foaming the composition either in the hand of the user, on the face or body. Examples of post-foaming shave gels are found in U.S. Pat. Nos. 7,820,152 (McLaughlin et al.); 5,858,342 (Szymczak); 5,560,859 (Hartmann et al.); 5,500,211 (George et al.); and 5,326,556 (Barnet et al.). Examples of post-foaming soap may be found in U.S. Pat. No. 4,772,427 (Dawson et al.).

Some cleaning compositions may be dispensed in the form of post-foaming liquids onto a hard surface or onto a sponge. These compositions are likely to be liquids and not gels, and are exemplified in U.S. Pat. Nos. 5,962,396; 6,004,920; and 6,051,542 (each to Pollack, et. al).

However, it does not appear that hair styling gels are dispensed from aerosolized packages and there doesn't appear to be any reason to do so. Heretofore it is unknown to change the texture and feel of a hair styling gel in manufacturing and then by virtue of the dispensing means stabilize a particular texture.

Accordingly, it is desirable to find new ways to stably store and dispense hair care gel compositions. Equally desirable is to explore hair care dispensing systems that may provide a more easily spread and/or more aesthetically appealing hair care styling product for the consumer.

### BRIEF SUMMARY OF THE INVENTION

The present invention is in general an aerated gel delivery system in combination with a hair styling composition.

In a preferred embodiment of the present invention, an aerated hair styling gel is dispensed in the form of an aerated gel from a bag-on-valve (bag-in-can) dispensing package.

In a preferred embodiment of the present invention, a hair styling gel is entrained with a sufficient amount of air during mixing to produce a gel having a specific gravity of less than about 0.995 g/cm³ prior to packaging in a bag-on-valve (bag-in-can) package having an impervious bag liner.

In a preferred embodiment of the present invention, an aerated hair styling gel minimally comprising synthetic fixative polymer, water and entrained air, maintains aerated stability in a bag-on-valve (bag-in-can) package and is dispensed as an aerated gel having a lower specific gravity than typical hair styling gels found packaged in cosmetic tubes or in jars. The bag-on-valve (bag-in-can) package comprises an impervious bag that maintains the stability and aerated physical form of the hair gel composition.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention. In particular, there are many variations possible for the hair styling gel compositions for use in the present pressurized delivery system. Such variations of particular ingredients in the hair styling gel are within the scope of the present invention.

The present invention is in general a pressurized product comprising an aerated hair styling gel and a bag-on-valve (bag-in-can) package. The present invention comprises a stable system of aerated gel packaged in a bag-on-valve package having an impervious liner that prevents the aerated gel from losing its entrained air. The aerated hair styling gel dispensed from the bag-on-valve package appears “whiter,” it feels lighter and fluffed in the user's hand, and it spreads more easily in the hands and on the hair of the user. Aerated gels packaged in cosmetic tubes are expected to lose their entrained air over time because the polyethylene or polypropylene packaging is permeably to air.

More specifically, the present invention is an aerated hair styling gel delivery system. That is, an aerated gel delivery system in combination with a hair styling gel, comprising:
a portable container having a gas impervious container wall;
a pressure source containing a pressurized gas disposed within said container;
a selectively operable valve assembly affixed to said container; and
an aerated hair styling composition in gel form disposed within said container, said hair styling composition minimally comprising synthetic fixative polymer and/or copolymer, water and entrained air.

The Aerated Hair Styling Gel Composition

The aerated hair styling gel composition for use in the present gel delivery system minimally comprises synthetic fixative polymer, water, and entrained air. What is meant by “synthetic fixative polymer” is that the polymer backbone is not present in nature but instead created synthetically (e.g. in a laboratory or manufacturing plant) by polymerization of vinylic monomers such as (meth)acrylates. Therefore, based on this definition, “synthetic fixative polymer” is meant to include polymers and copolymers such as polyvinylpyrrolidone (PVP) and vinyl pyrrolidone/vinyl acetate (VP/VA) since these are synthetic polymers derived by polymerization of vinyl monomers (e.g. vinyl pyrrolidone). The definition is meant to exclude starch derivatives such as carboxymethyl cellulose, because the term “synthetic” refers herein to the backbone of the hair fixative polymer and not to any synthetic modifications made to a naturally derived polymer backbone such as cellulose. Meaning, the synthetic fixative polymers by definition here have a synthetically derived repeating backbone and not a polymer backbone created in nature or by a biochemical process. Thus, the present composition minimally comprises a synthetic fixative polymer such as PVP. However, it is important to note that the present composition may also include starch and/or starch derivatives (which are fixative to the hair to some extent), as optional ingredients to augment the hair styling effects derived from the synthetic polymers and/or copolymers. Most of the preferred ingredients for use in the aerated hair styling gel composition of the present invention have been disclosed in the following references, each of which incorporated herein in its entirety by reference: U.S. Patent Application Publications 2009/0123405 (Sun et al.); 2009/0074697 (Huynh); and 2008/0102051 (Huynh et al.). All of the ingredients discussed below will have percentages given for the amount of these materials preferably incorporated into the aerated gel compositions of the present invention. In each and every case, the percent (%) given is meant as weight percent actives, and is based on the total weight of the composition. For example, incorporation of 20% as is of a 40% active polymer results in a weight percent of 8 wt. %.

The polymer or copolymer that forms an essential component of the aerated gel compositions of the present invention may be selected from the group consisting of nonionic, anionic, zwitterionic or amphoteric or cationic synthetic homopolymers, and copolymers. For example, nonionic polymers of use in the present invention include polyvinylpyrrolidone, copolymers of N-vinylpyrrolidone and vinyl acetate, and/or vinyl propionate, polyvinylcaprolactam, polyvinylamides and salts thereof, and copolymers of vinylpyrrolidone and dimethylaminoethyl methacrylate, terpolymers of vinylcaprolactam, vinylpyrrolidone and dimethylaminoethyl methacrylate, polysiloxanes and the like.

Anionic polymers of use in the present invention include vinyl acetate/crotonic acid, vinyl acetate/acrylate and/or vinyl acetate/vinyl neodecanoate/crotonic acid copolymers, sodium acrylate/vinyl alcohol copolymers, sodium polystyrenesulfonate, ethyl acrylate/N-t-butylacrylamide/acrylic acid copolymers, vinylpyrrolidone/vinylacetate/itaconic acid copolymers, acrylic acid/acrylamide copolymers and/or sodium salts thereof, homo- and/or copolymers of methacrylic acid and/or salts thereof, and acrylate/hydroxyacrylate, octylacrylamide/acrylate or methacrylate and/or butyl acrylate/N-vinylpyrrolidone copolymers or polystyrenesulfonates.

Amphoteric polymers that find use in the present invention include copolymers of N-octylacrylamide, methacrylic acid and tert-butylaminoethyl methacrylate of the “amphomer” type, copolymers of methacryloylethylbetaine an alkylmethacrylates of the “yukaformer” type, copolymers of monomers containing carboxyl groups or sulfone groups, for example methacrylic acid and itaconic acid, with basic group-containing monomers such as mono- or dialkylaminoalkyl methacrylates and/or mono- and dialkylaminoalkyl methacrylamides, copolymers of N-octylacrylamide, methyl methacrylate, hydroxypriopyl methacylate, N-tert-butylaminoethyl methacrylate and acrylic acid.

Cationic polymers that may find use in the present invention include vinylpyrrolidone/vinylimidazolium methochloride copolymers, quaternized vinylpyrrolidone/diakylaminoalkyl methacrylate copolymers, such as hydroxyethylcellulose/dimethylalkylammonium chloride copolymers, and terpolymers of vinylcaprolactam/vinylpyrrolidone with dimethylaminoethyl methacrylate or vinylimidazolium methochloride and acrylamido copolymers.

That being said, the preferred polymers for use in the present invention include but are certainly not limited to vinyl pyrrolidone/dimethylaminoethyl methacrylates copolymer, vinyl acetate/crotonates/vinyl neodecanoate copolymer, octyl acrylamide/acrylates/butylaminoethyl methacrylate copolymer, vinyl acetate/crotonates, polyvinylpyrrolidone (PVP), polyvinylpyrrolidone/vinyl acetate copolymer, PVP acrylates copolymer, vinyl acetate/crotonic acid/vinyl proprionate, acrylates/acrylamide, acrylates/octylacrylamide, acrylates copolymer, acrylates/hydroxyacrylates copolymer, and alkyl esters of polyvinylmethylether/maleic anhydride, diglycol/cyclohexanedimethanol/isophthalates/sulfoisophthalates copolymer, vinyl acetate/butyl maleate and isobornyl acrylate copolymer, vinylcaprolactam/PVP/dimethylaminoethyl methacrylate, vinyl acetate/alkylmaleate half ester/N-substituted acrylamide terpolymers, vinyl caprolactam/vinylpyrrolidone/dimethylaminoethyl methacrylates copolymer, vinyl caprolactam/vinylpyrrolidone/methacryloamidopropyl trimethylammonium chloride terpolymer, methacrylates/acrylates copolymer/amine salt, polyvinylcaprolactam, polyurethanes, polyquaternium-4, polyquaternium-10, polyquaternium-11, and polyquaternium-46.

The most preferred polymers for use in the compositions of the present composition are various ratios and amounts of polyvinylpyrrolidone, polyvinylpyrrolidone/vinyl acetate copolymer, vinyl pyrrolidone/dimethylaminoethyl methacrylates copolymer and vinyl caprolactam/vinyl pyrrolidone/dimethylaminoethyl methacrylates copolymer. The preferred total amount of synthetic fixative polymers is from about 5% to about 70%, based on the total weight of the gel composition, consisting of either one of these, or combinations thereof. More preferred is to incorporate from about 5% to about 50% by weight actives of one or more of these synthetic fixative polymers, based on the total weight of the gel composition. The most preferred polymers for use in the present compositions include polyvinylpyrrolidone 20% aqueous solution, or polyvinylpyrrolidone powder, sold under the trade names of Luviskol K-90® and Luviskol-PVP® respectively from BASF, polyvinylpyrrolidone/vinylacetate copolymer (PVP/VA-copolymer), sold under the trade name Luviskol VA-73W® from BASF, vinyl pyrrolidone/dimethylaminoethyl methacrylate copolymer (VP/DMAPA-copolymer), sold under the trade name of Styleze CC-10® from ISP, vinyl caprolactam/vinyl pyrrolidone/dimethylaminoethyl methacrylate copolymer with 0.77% ethanol, sold under the trade name of Advantage®-S Solution from ISP, and vinyl caprolactom/vinyl pyrrolidone/dimethylaminoethyl methacrylate copolymer with 63% ethanol, sold under the trade name of Advantage® LC-A from ISP.

The compositions of the present invention may optionally include starch, modified starches and/or other cellulosic material or combinations of these, for modifying the quality of the dried product on the hair and for modifying the viscosity of the actual composition. For example, of use in the compositions of the present invention include the celluloses, cellulose derivatives, cellulose gums, ethoxylated celluloses, starch or gums, guar gum, cationic cellulose derivatives such as guar hydroxypropyl trimonium chloride, xanthan gum, hydroxypropyl guar, karaya gum, and the like. Also of use in the present invention are pregelatinized crosslinked starch derivatives, including hydroxypropyl distarch phosphate, as described in U.S. Patent Application Publication US2005/0191264 and incorporated herein by reference. Preferred cellulosic materials of use in the compositions of the present invention include nonionic or cationic saccharides such as cellulose ethers including methyl cellulose, carboxymethyl cellulose, hydroxy propyl methyl cellulose, hydroxyethyl cellulose, hydroxypropyl cellulose, and ethyl hydroxyethyl cellulose, dextrans obtained from Sigma, Kitamer PC, a chitosan carboxylate and Kytamer L, a chitosan lactate obtained from Amerchol, Gafquat HS-100, Polyquaternium-28 from International Specialties, polyquaternium4, polyquaternium-10, sodium alginate, agarose, amylopectins, amyloses, arabinans, arabinogalactans, arabinoxylans, carrageenans, gum arabic, cellulose derivatives such as methylcellulose, hydroxypropylmethylcellulose, hydroxyethyl cellulose, carboxymethylcellulose, carboxymethylguar gum, carboxymethyl(hydroxypropyl)guar gum, hydroxyethylguar gum, hydroxypropylguar gum, cationic guar gum, chondroitins, chitins, chitosans, cocodimonium hydroxypropyl oxyethyl cellulose, colominic acid [poly(N-acetyl-neuraminic acid], corn starch, curdlan, dermatin sulfate, furcellarans, dextrans, cross-linked dextrans known as dextranomer (Debrisan), dextrin, emulsan, flaxseed saccharide (acidic), galactoglucomannans, galactomannans, glucomannans, glycogens, guar gum, or hydroxyethylstarch, hydroxypropylstarch, hydroxypropylated guar gums, gellan gum, glucomannans, gellan, gum ghatti, gum karaya, gum tragacanth (tragacanthin), heparin, hyaluronic acid, inulin, keratan sulfate, konjac mannan, laminarans, laurdimonium hydroxypropyl oxyethyl cellulose, liposan, locust bean gum, mannans, nigeran, nonoxylnyl hydroxyethyl cellulose, okra gum, oxidized starch, pectic acids, pectins, polydextrose, potato starch, protopectins, psyllium seed gum, pullulan, sodium hyaluronate, steardimonium hydroxyethyl cellulose, raffinose, rhamsan, tapioca starch, welan, levan, scleroglucan, stachyose, succinoglycan, wheat starch, xanthan gum, xylans, xyloglucans, and mixtures thereof. Microbial saccharides can be found in the fourth edition of Kirk-Othmer Encyclopedia of Chemical Technology, Fourth Edition. Vol. 16, John Wiley and Sons, NY pp. 578-611, 1994. Complex carbohydrates can be found in the fourth edition of Kirk-Othmer Encyclopedia of Chemical Technology, Fourth Edition. Vol. 4, John Wiley and Sons, NY pp. 930-948, 1994. Of most particular use in the present invention is hydroxyethylcellulose and a modified corn starch from National Starch found under the trade name Amaze®. Most preferred for use in the compositions of the present invention is from about 0.01% to about 10% total cellulosic and starch materials, by weight in the composition. Most preferred is to use hydroxyethylcellulose and Amaze® modified corn starch from National Starch, together in the composition at a total of from about 0.1% to about 5% by weight.

The compositions of the present invention may also include a thickening polymer, such as a high molecular weight polyacrylate. Thickeners can be utilized alone or in combination so long as the chosen thickeners are compatible with the hair styling composition. Thickeners may include, but are not limited to, acrylic acid homopolymers under the Carbopol® trade name from BF Goodrich, acrylates/C10-30 alkyl acrylate crosspolymer (Carbopol® 1342, 1382, Pemulins® TR-1 and TR-2 from BF Goodrich), Acrylates/Steareth-20 Itaconate Copolymer, Acrylates/Ceteth-20 Itaconate Copolymer from National Starch, Bentonite, PVM/MA Decadiene Crosspolymer from International Specialties Products, Acrylates/steareth-20 methacrylate copolymer, Acrysol® ICS-1 from Rohm and Haas Co., acrylamide/sodium acrylate copolymer, Hostacerin® PN 73, Hoecsht AG., acrylate copolymer (Antil® 208) supplied by Degussa-Goldschmidt, acrylic acid/acrylonitrogens copolymer (Hypan® SA-100H, SR-150H) supplied by Lipo, Acrylic/acrylate copolymer (Carboset® 514, 515, 525, XL-19, XL-19X2, XI-28, XL40, 526) supplied by BF Goodrich, Ammonium acrylateslacrylonitrogens copolymer (Hypan® SS-201) from Lipo, Quaternium-18 Bentonite, Sodium salt of crosslinked poly(acrylic acid) under the tradenames PNC® 430, PNC® 410, PNC® 400 from 3V, Stearalkonium Bentonite, Claytone, supplied by Southern Clay, Quaternium-18 Hectorite (Bentone 38), Stearalkonium Hectorite (Bentone 27) supplied by Rheox, acrylamide/sodium acrylate copolymer (Hostacerin PN 73) supplied by Hoechst, Poly(acrylic acid) known as Carbopol® 400 series (BF Goodrich) or Aquatreat® (Alco 3V), polyquaternium-18 (Mirapol® AZ-1) from Rhone Poulenc, polyquaternium-27, polyquaternium-31, polyquaternium-37, trihydroxystearin (Thixcin from Rheox; Flowtone from Southern Clay), Dimethylaminoethyl methacrylamide and acrylamide copolymer (Salcare® SC63 from Ciba Specialties), Acrylic polymer cationic thickening agents (Synthalen® CR and its related compounds) from 3V Sigma. Other thickeners and polymers can be found in the “The Encyclopedia of Polymers and Thickeners for Cosmetics,” Cosmetics and Toiletries, Lochhead, R., pp. 95-138, Vol. 108, (May 1993). Thickeners, if desired in the composition, may be incorporated at from about 0.01% to about 50% by weight active polymer.

The compositions of the present invention may also include petrolatum or other waxes and oils. Petrolatum is a mixture of hydrocarbons that finds use in various personal care products. Preferred petrolatum includes white petrolatum USP, petrolatum USP, mineral jelly and ointment base. The melting point ranges of the preferred petrolatum for use in the present invention may be from about 80° F. to about 135° F. Most preferred is to use UltraPure® Liquid Petrolatum USP from Ultra Chemicals that has a melting point range of about 105°-115° F., or various grades (ranging in color) of Penreco® Petrolatum USP having melting point ranges around 122°-135° F. The compositions of the present invention may also contain an emulsifying wax and/or oil. Such materials include the non-limiting examples of bees' wax, candelilla wax, carnauba wax, emulsifying wax (for example Polawax® from Croda) and Jojoba, safflower, canola (tribehenin), tallow, lard, palm, castor, sunflower seed, or soya bean oil oils, or hydrogenated derivatives thereof. Most preferred is to incorporate Polawax®, jojoba oil, safflower oil, tribehenin, and/or hydrogenated castor oil, singularly or in any combination. When desired in the present aerated gel compositions, any combination of these materials may be used at from about 0.1% to about 5% by weight of the total composition. Most preferred is to use (individually or in combination) petrolatum, wax and/or oil at from about 1% to about 3% by weight in the composition.

The aerated hair styling gel compositions for use in the present delivery system may also include a fatty alcohol, polyols, and/or other substances.

Fatty alcohols that may find use in the compositions of the present invention include naturally derived and synthetic materials. These are high molecular weight straight or branched chain primary alcohols. Most preferred for use in the present invention include lauryl (C12), myristyl (C14), cetyl or palmityl (C16), stearyl (C18), oleyl (C18-unsaturated) and linoleyl (C18-polyunsaturated) alcohols, or combinations thereof. Ceteryl alcohol is also preferred and is a mixture of cetyl and stearyl alcohols. The fatty alcohol in the present invention may be incorporated at from about 0.01% to about 5% by weight in the composition.

Polyols of use in the present invention include but are not limited to straight-chain molecules having carbon chain length of from about 3 to about 8, most preferably from about 5 to about 6, with the number of hydroxyl groups from 3 to about 8, most preferably from about 5 to about 6. “Polyols” in the context of the present invention are understood to be substances with 3 or more hydroxyl groups (that is, including triols but excluding diols). For example, of use in the present invention are straight chain polyols selected from the group consisting of erythritol, glycerol (i.e. glycerin), mannitol, sorbitol and xylitol. The polyols that find use in the present invention may be natural or synthetically derived, and may also include cyclic, or combinations of straight chain and cyclic structures. For example, isomalt, lacitol, maltitol, and various HSH's (hydrogenated starch hydrosylates) may find use in the present invention. Out of all the sugar alcohols and simple polyols, most preferable is to use mannitol or sorbitol (both C₆H₁₄O₆) or combinations of the two. Polyols that contain, in addition to the requisite minimum (3) hydroxyl groups, other functional groups in the molecule such as aldehydes, ketones, carboxylate, thiols, etc., are also of use in the present invention. For example, methyl 2,5,6-trihydroxyhexanoate, or other functionalized polyols may find use herein. Additionally, natural sugar monosaccharides that are in equilibrium mixture of open straight chain form and cyclic aldol or ketol form may also find use in the present invention. Many small organic molecules, or mixtures of them, may find use in the compositions of the present invention provided they minimally have three or more hydroxyl groups. The polyol or polyols is/are preferably incorporated in the compositions of the present invention at from about 5% to about 15% by weight in the composition. Most preferred is to incorporate glycerin at from about 5% to about 15% by weight based on the total weight of the composition.

Also of use as optional ingredients in the aerated gel compositions of the present invention are emulsifiers. Emulsifiers for use in cosmetic applications are amply listed in McCutcheon's Emulsifiers and Detergents. Many suitable emulsifiers are nonionic esters or ethers comprising a polyoxyalkylene moiety, especially a polyoxyethylene moiety, often containing from about 2 to 80, and especially 5 to 60 oxyethylene units, and/or that contain a polyhydroxy compound such as glycerol or sorbitol or other alditol as hydrophilic moiety. The hydrophilic moiety can contain polyoxypropylene. The emulsifiers additionally contain a hydrophobic alkyl, alkenyl or arylalkyl moiety, normally containing from about 8 to 50 carbons. The hydrophobic moiety can be either linear or branched and may be saturated, mono-unsaturated or polyunsaturated, and is optionally fluorinated. The hydrophobic moiety can comprise a mixture of chain lengths, for example those deriving from tallow, lard, palm oil, sunflower seed oil or soya bean oil. Such nonionic materials can also be derived from a polyhydroxy compound such as glycerol or sorbitol or other alditols. Examples of such substances include, but are not limited to, oleth-20, ceteareth-10 to -25, ceteth-10-25, steareth-10-25 (i.e. C₁₆ to C₁₈ alcohols ethoxylated with 10 to 25 ethylene oxide residues) and PEG-15-25 stearate or distearate. Other suitable examples include C₁₀-C₂₀ fatty acid mono, di- or tri-glycerides. Further examples include C₁₈-C₂₂ fatty alcohol ethers of polyethylene oxides (8- to 12-EO). Other examples of useful emulsifiers are fatty acid mono or possibly diesters of polyhydric alcohols such as glycerol, sorbitol, erythritol or trimethylolpropane. The fatty acyl moiety is often from C₁₄ to C₂₂ and is saturated in many instances, including cetyl, stearyl, arachidyl and behenyl. Examples include monoglycerides of palmitic or stearic acid, sorbitol mono or diesters of myristic, palmitic or stearic acid, and trimethylolpropane monoesters of stearic acid. Another usable class of emulsifiers comprises dimethicone copolymers, namely polyoxyalkylene modified dimethylpolysiloxanes. The polyoxyalkylene group is often a polyoxyethylene (POE) or polyoxypropylene (POP) or a copolymer of POE and POP. The copolymers often terminate in C₁ to C₁₂ alkyl groups. Such suitable emulsifiers and co-emulsifiers are widely available under many trade names and designations including Abil®, Arlacel®, Brij®, Cremophor®, Dehydrol®, Dehymuls®, Emerest®, Lameform®, Pluronic®, Prisorine®, Quest PGPR®, Span® Tween®, SF1228, DC3225C and Q2-5200. Any of these materials may find use in the gel composition of the present invention and are incorporated, either singly or as mixtures, at from about 0.01% to about 10% by weight actives, based on the total weight of the gel composition.

Also, the compositions of the present invention may include “oil-soluble film former” polymers. Non-limiting examples of the oil-soluble film former include polymethylsilsesquioxanes; acrylic fluorinated emulsion film formers, such as Foraperle® film formers (e.g., Foraperle® 303 D available from Elf Atochem); GANEX® copolymers, such as butylated PVP, PVP/Hexadecene copolymers, PVP/Eicosene copolymers, and tricontanyl; Poly-(vinylpyrrolidone/diethylaminoethyl methacrylate) copolymers and PVP/Dimethylaminoethylmethacrylate copolymers such as Copolymer 845 available from I.S.P.; Resin ACO-5014 (Imidized IB/MA copolymer); other PVP based polymers and copolymers; silicone gums; cyclomethicone copolymers and dimethicone crosspolymers, such as Dow Corning® 2-9040 and those disclosed in U.S. Pat. No. 5,654,362, the disclosure of which is hereby incorporated by reference; trimethyl siloxysilicates such as SR 1000, 554230, and SS4267 available from GE Silicones; alkyl cycloalkylacrylate copolymers, such as those disclosed in WO 98/42298, the disclosure of which is hereby incorporated by reference; Mexomere® film formers and other allyl stearate/vinyl acetate copolymers; polyolprepolymers, such as PPG-12/SMDI copolymer, also called Poly-(oxy-1,2-ethanediyl), α-hydro-ω-hydroxy-polymer with 1,1′-methylene-bis-(4-isocyanatocyclohexane) available from Barnet; and Avalure® AC Polymers (Acrylates Copolymer) and Avalure® UR polymers (Polyurethane Dispersions), available from BFGoodrich. Preferred for use in the present invention is the already mentioned tricontanyl PVP copolymer sold under the trade name Ganex® WWP-660 from ISP. Any combination of the Ganex® V/WP grades are excellent film formers for the present invention and provide water and wear resistance and a moisture barrier to the set hair. The oil-soluble film former polymer may be present in the composition from about 0.1% to about 5% by weight.

Also optional to the compositions of the present invention is the addition of a chelant. Chelants that may find use herein include but are not limited to phosphates (organic and inorganic), NTA, the various ethylenediaminetetraacetic acid (EDTA) derivatives, and lower molecular weight polyacrylates. For example, the present invention may include disodium-, trisodium- or tetrasodium-EDTA, various salts of NTA, phosphate esters, or Acusol® 445 from Rohm and Haas. The chelant may be present in the composition from about 0.001% to about 1% by weight.

The compositions of the present gel delivery system may also include an alkaline agent, particular if used for neutralizing one or more acidic polymers to a more neutral pH to generate thickening. Such alkaline materials include, but are not limited to, hydroxides, carbonates, bicarbonates, sesquicarbonates, amines, and/or alkanolamines such as monethanolamine, diethanolamine, triethanolamine, and/or amino methyl propanol (AMP). If used in the gel compositions herein, from about 0.01% to about 10% by weight active alkaline agent may be used to adjust pH and viscosity.

In addition to adding water, a necessary component to the present gel composition, alkanol solvents such as ethanol, propanol, isopropanol, butanol, and the like, or mono- and/or dialkylether solvents such as propylene glycol monomethyl ether or ethylene glycol monophenyl or monobutyl ether, or ethylene glycol and/or propylene glycol, and the like, may find use in the present gel compositions. Additionally, solvents such as acetone, methyl acetate, d-limonene and the like may find use in the present invention. The water in the composition may be present from about 1% to about 90%, and more preferably at least about 40%, whereas additional solvents may be present at less than about 20% by weight.

Air is also a required ingredient in the aerated hair styling gel composition herein. Air is preferably entrained into the gel mixture during the mixing of the other ingredients mentioned above. Entrainment of air may be achieved by simple stirring, vigorous stirring, shaking, agitating, or even homogenizing the gel mixture at any stage in the formulation. For example, the mixing vessel may be charged with just water and an emulsifier such as an ethoxylated alcohol. Then the mixing in the vessel may be increased to high speed, creating a deep enough vortex in the liquid such that air is sucked down and entrained into the surfactant/water mixture. During this vortexing, the additional ingredients of the gel composition may be added sequentially as necessary. Alternatively, air entrainment may be a separate and deliberate step after the gel composition is completely formulated. For example, after the last ingredient is added to the composition and it is mixed in, air may be entrained by inserting an air source (a dip-tube, perforated wand or manifold or other “bubbler”) or by increasing the stirring until a vortex is obtained, and/or by lowering a homogenizer into the composition. Also, air could be entrained into the gel while the finished gel composition is being pumped over to filling lines for packaging, such as by combining an air stream with a product stream en route to the filling lines. Also, air could be entrained at the moment of filling, such as by configuring an air line to impinge directly into the outlet tip of the metering filler. The point being, air is entrained anywhere and at anytime in the process up until when the pressured packaging system is sealed and no more change can be done to the composition.

Since air is difficult to measure by weight or by volume, the amount of air preferably entrained into the gel composition to create an “aerated hair styling gel” is that amount of air sufficient to produce an end specific gravity of the gel composition of less than 0.9950 grams/cm³. Typically a hair styling gel that comprises water, synthetic fixative polymer, polyacrylate thickener, alkaline pH adjusting agent, and some emulsifier(s), will have an end specific gravity close to 1.0000 grams/cm³, more likely between about 0.9950-1.0000 grams/cm³ and most likely about 0.9980 grams/cm³. In order to produce an aerated gel having consumer acceptable feel and appearance, sufficient air is preferably entrained into the composition to lower the specific gravity to less than 0.9950 grams/cm³. What is meant by the “end specific gravity” is the specific gravity measured at the time of packaging. That is, when nothing more can be done to the composition because it is being filled into the pressuring packaging. The inventiveness of the present aerated gel delivery system is that the aeration remains stable in the preferred bag-on-valve packaging used in the present system. Thus, the amount of aeration, as measured and confirmed by a specific gravity measurement prior to packaging, is optimized just prior to packaging. With this definition and description in mind, the end specific gravity of the aerated gel composition just prior to packaging is adjusted by aeration to be less than 0.9950 grams/cm³. The resulting aerated hair styling gel of the present invention is whiter in appearance than typical hair styling gels marketed in screw lid “tubs,” (most of which are purposely crystal clear), and the product, being lighter, (as evidenced by the lower specific gravity) gives more individual uses for the consumer than a similar weight package of non-aerated gel. As a final point, it is important to distinguish the present inventive “aerated gel” with a true “foam.” Foam has a specific gravity that is considerably less than the preferred specific gravity of the present invention. Foams may have specific gravity of less than 0.2 grams/cm³ whereas the present inventive gel preferably has a specific gravity of less than grams/cm³, but impliedly greater than about 0.8000 grams/cm³ because the present invention is not foam or a foam delivery system.

Finally, the aerated gel composition may include typical optional ingredients found in hair care compositions and personal care products in general such as fragrance, pigments and/or colorants to give color to the gel product, hair dyes to temporarily or permanently color the hair styled by the product, UV-absorbing ingredients (to protect dyed hair and/or protect the composition, antioxidants, antistatic, and/or microbial growth preventatives, (i.e. preservatives).

The Bag-on-Valve (Bag-in-Can) Packaging

The preferred delivery system of the present invention includes the bag-on-valve (bag-in-can) system, such as the systems available from Aptar or the ABS® bag-in-can system available from CCL Container. These preferred systems are hand held, portable, and completely self-contained dispensers. The bag-in-valve system separates the product from the pressurizing agent with a hermetically sealed, multi-layered laminated “bag”. The bag maintains total formulation integrity, dispensing only pure product as opposed to product mixed with the gaseous propellant. This maintains the total product integrity, such as maintaining the air previously entrained into gel mixtures and potentially extends the shelf life of a gel product. The bag-on-valve dispensers are adequately disclosed in the following references, each of which is incorporated in its entirety by reference: U.S. Pat. Nos. 8,002,000; 7,730,911; 7,523,767; and 7,124,788 (each to Pericard, and assigned to Precision Valve Corp.); and 7,913,877 (Neuhalfen, assigned to AptarGroup). These references disclose the materials of construction, the specifications, and methods of manufacturing and filling.

The typical multi-layered laminated bag comprises polyethylene terephthalate (PET), foil, nylon, and/or polypropylene (PP) layers, and is the preferred laminate bag for use herein. However, other combinations of these and other material layers may be envisioned for the laminated bag provided the bag is impervious to air migration. Such impermeability is required so that the aerated gel composition maintains its lowered specific gravity. For the bag-on-valve (BOV) system, the bag is purchased already attached to the male or female selectively operable valve assembly. A valve assembly includes the valve body, spring, piston, mounting cup, internal gasket, and stem (for male). Selectively operable refers to on-demand actuation, i.e. by depressing the spring therein. A dip tube is not required for BOV systems since the internal squeezing of the bag by the pressurized gas brings the gel up to the valve. Most preferred are any of the EP Systems™ (1″ BOV) available from Aptar. Such a 1″ valve cup assembly can be crimped onto any appropriately dimensioned aluminum or steel can, and subsequently fitted with a gel delivery spout. The most preferred spout for use on the inventive system here is the Allegra™ spout from Aptar specifically designed for gel delivery.

The typical gas used to pressurize the space in between the bag and the can may be pressurized air and/or nitrogen, although other inert gasses are possible. The outside container comprises gas impervious walls and is typically made of a metal such as aluminum or tinplate steel, or plastic. The outer container may seamless, single piece or multiple pieces (e.g. 3-piece tin plate can). The preferred can is the 1-piece aluminum can.

Alternatively, but less preferably, this invention can employ a piston barrier package that also assures separation of the aerated hair styling gel from the propellant or pressurizing agent. Such a system may also help maintain the purity and integrity of the gel throughout its consumer life span and ensure that the gel remains aerated with low specific gravity. Such a system is available from CCL Container and it provides efficient high viscosity delivery (such as for shave gels). The system provides smooth controlled and uniform discharge. In a piston barrier package, a piston is disposed in a preferred seamless tubular package between the propellant and the product. As the nozzle is activated, the piston is activated by the propellant pressure in the lower part of the container, to push out the product through the nozzle. A preferred system having seamless aluminum can construction eliminates propellant bypass tendency common with other metal containers. Such systems feature a specially engineered gassing hole at the bottom for improving sealing during gassing and plugging operations.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

We claim:

1. An aerated gel delivery system in combination with a hair styling gel, comprising:
a) a portable container having a gas impervious container wall;
b) a pressure source containing a pressurized gas disposed within said container;
c) a selectively operable valve assembly affixed to said container; and
d) an aerated hair styling composition in gel form disposed within said container, said hair styling composition comprising synthetic fixative polymer and/or copolymer, water and entrained air; and
wherein said aerated hair styling gel composition is entrained with sufficient air such that the gel composition prior to packaging in said container has a specific gravity of less than 0.9950 grams/cm³ but greater than about 0.8000 grams/cm³, and
wherein said aerated gel is dispensed by the pressurized gas compressing on a product bag of a bag-on-valve package containing said aerated hair styling gel composition so that the aerated gel effluent contains no portion of said pressurized gas.

2. The system of claim 1, wherein said synthetic fixative polymer comprises a mixture a polyvinylpyrrolidone and vinyl pyrrolidone/vinyl acetate copolymer.

3. The system of claim 1, wherein said composition further comprises a polyacrylate thickener.

4. The system of claim 1, wherein said composition further comprises an ethoxylated alcohol emulsifier.

5. The system of claim 1, wherein said composition further comprises a polyol.

6. The system of claim 1, wherein said composition further comprises a starch and/or starch derivative.

7. The system of claim 1, wherein said product bag comprises a laminate of polymer and foil layers.

8. The system of claim 1, wherein said product bag comprises a laminate of polyethylene terephthalate, foil, nylon, and polypropylene layers.

9. The system of claim 1, wherein said selectively operable valve assembly comprises a 1″ diameter valve cup.

10. An aerated gel delivery system in combination with a hair styling gel, comprising:
a) a portable container having a metal container wall;
b) a pressure source containing a pressurized gas disposed within said container;
c) a selectively operable valve assembly affixed to said container; and
d) an aerated hair styling composition in gel form disposed within said container, said hair styling composition comprising polyvinylpyrrolidone, vinyl pyrrolidone/vinyl acetate copolymer, polyacrylate, polyol, ethoxylated alcohol, water and sufficient entrained air such that the gel composition prior to packaging in said container has a specific gravity of less than 0.9950 grams/cm³ but greater than about 0.8000 grams/cm³, and
wherein said aerated gel is dispensed by the pressurized gas compressing on a product bag of a bag-on-valve package containing said aerated hair styling gel composition so that the aerated gel effluent contains no portion of said pressurized gas.
